(19) United States
(12) Patent Application Publication
(10) Pub. No.: US 2024/0402143 A1
(43) Pub. Date: Dec. 5, 2024

(54) GAS ANALYZING METHOD, AND GAS ANALYZING SYSTEM

(71) Applicant: Panasonic Intellectual Property Management Co., Ltd., Osaka (JP)

(72) Inventor: Atsuo NAKAO, Nara (JP)

(21) Appl. No.: 18/687,214

(22) PCT Filed: Sep. 5, 2022
(86) PCT No.: PCT/JP2022/033246

§ 371 Date: Feb. 27, 2024

(30) Foreign Application Priority Data
Sep. 7, 2021 (JP) 2021-145540

(51) Int. Cl. G01N 33/00; G01N 5/02

(52) U.S. Cl. CPC G01N 33/0027; G01N 5/025

(57) ABSTRACT

A gas analyzing method includes obtaining, dividing, selecting, extracting, and analyzing. In the obtaining, signal is obtained from a sensor exposed to sample gas under a condition where molecules contained in the sample gas is more readily adsorbed to the sensor in a second period than in a first period and a third period in a measurement period for the exposure. In the dividing, each of the second and third periods is divided into divided sections. In the selecting, an extraction section is selected from a part of the divided sections. In the extracting, feature of the signal in the extraction section is extracted. In the analyzing, analysis on the molecules contained in the sample gas is performed based on the extracted feature among features of the signal in the measurement period, and a result of the analysis is outputted.

### CROSS-REFERENCE OF RELATED APPLICATIONS

This application is the U.S. National Phase under 35 U.S.C. § 371 of International Patent Application No. PCT/JP2022/033246, filed on Sep. 5, 2022, which in turn claims the benefit of Japanese Patent Application No. 2021-145540, filed on Sep. 7, 2021, the entire disclosures of which Applications are incorporated by reference herein.

### TECHNICAL FIELD

The present disclosure relates to a gas analyzing method and a gas analyzing system.

### BACKGROUND ART

A gas is analyzed based on a signal obtained from a sensor exposed to the gas, for example. Patent Literature (PTL) 1 discloses a method of identifying an analysis target using data on a pulsed signal resulting from detecting the analysis target. This method uses features of the pulsed signal, such as intensity, wavelength, intensity ratio, and kurtosis.

### CITATION LIST

### Patent Literature

[PTL 1] WO 2018/207524

### SUMMARY OF INVENTION

### Technical Problem

For an analysis of molecules contained in a gas using a sensor, the presence of a molecule different from a target molecule may decrease the analytical accuracy.

In response to this, the present disclosure provides a gas analyzing method that is capable of increasing accuracy in analyzing molecules contained in a gas.

### Solution to Problem

In accordance with an aspect of the present disclosure, a gas analyzing method using a sensor that outputs a signal responsive to molecular adsorption includes: obtaining a signal outputted from the sensor that is exposed to a sample gas in a measurement period including a first period, a second period following the first period, and a third period following the second period, under a condition where a molecule contained in the sample gas is more readily adsorbed to the sensor in the second period than in the first period and the third period; dividing at least one of the second period or the third period into a plurality of divided sections, and selecting, as at least one extraction section, a part of the plurality of divided sections; extracting at least one feature of the signal outputted in the at least one extraction section; and performing, using a pre-trained logical model for one of qualitative determination or quantitative determination of the molecule contained in the sample gas, one of qualitative analysis or quantitative analysis on the molecule contained in the sample gas, based on only the at least one feature extracted in the extracting among features of the signal in the measurement period, and outputting an analysis result of the performing of the one of qualitative analysis or quantitative analysis.

In accordance with another aspect of the present disclosure, a gas analyzing method using a plurality of sensors each of which outputs a signal responsive to molecular adsorption, the plurality of sensors exhibiting mutually different molecular adsorption behaviors includes: obtaining a plurality of signals outputted from each of the plurality of sensors that are exposed to a sample gas in a measurement period including a first period, a second period following the first period, and a third period following the second period, under a condition where a molecule contained in the sample gas is more readily adsorbed to the plurality of sensors in the second period than in the first period and the third period; extracting a plurality of features from each of the plurality of signals; and performing analysis to determine, using a pre-trained logical model for determining a concentration of a predetermined molecule contained in the sample gas, the concentration of the predetermined molecule contained in the sample gas, based on the plurality of features extracted in the extracting, and outputting an analysis result of the performing of analysis.

In accordance with still another aspect of the present disclosure, a gas analyzing system includes: a sensor that outputs a signal responsive to molecular adsorption; an exposer that exposes the sensor to a sample gas in a measurement period including a first period, a second period following the first period, and a third period following the second period, under a condition where a molecule contained in the sample gas is more readily adsorbed to the sensor in the second period than in the first period and the third period; an obtainer that obtains the signal outputted from the sensor in the measurement period; a selector that divides at least one of the second period or the third period into a plurality of divided sections, and selects, as at least one extraction section, a part of the plurality of divided sections; an extractor that extracts at least one feature of the signal outputted in the at least one extraction section; a memory that stores a pre-trained logical model for one of qualitative determination or quantitative determination of the molecule contained in the sample gas; and an analyzer that performs, using the pre-trained logical model, one of qualitative analysis or quantitative analysis on the molecules contained in the sample gas, based on only the at least one feature extracted by the extractor among features of the signal in the measurement period, and outputs an analysis result of the one of qualitative analysis or quantitative analysis.

In accordance with still another aspect of the present disclosure, a gas analyzing system includes: a plurality of sensors each of which outputs a signal responsive to molecular adsorption, the plurality of sensors exhibiting mutually different molecular adsorption behaviors; an exposer that exposes the plurality of sensors to a sample gas in a measurement period including a first period, a second period following the first period, and a third period following the second period, under a condition where a molecule contained in the sample gas is more readily adsorbed to the plurality of sensors in the second period than in the first period and the third period; an obtainer that obtains a plurality of signals outputted from each of the plurality of sensors in the measurement period; an extractor that extracts a plurality of features from each of the plurality of signals; a memory that stores a pre-trained logical model for determining a concentration of a predetermined molecule contained in the sample gas; and an analyzer that performs analysis to determine, using the pre-trained logical model, the concentration of the predetermined molecule contained in the sample gas, based on the plurality of features extracted by the extractor, and outputs an analysis result of the analysis.

### Advantageous Effects of Invention

The present disclosure enables an increase in the accuracy in analyzing molecules contained in a gas.

### BRIEF DESCRIPTION OF DRAWINGS

FIG. 1 is a schematic block diagram illustrating a configuration of a gas analyzing system according to Embodiment.

FIG. 2 is a schematic diagram illustrating an example of a configuration of an exposer according to Embodiment.

FIG. 3 is a schematic block diagram illustrating a configuration of a gas analyzing system according to a variation of Embodiment.

FIG. 4 is a flowchart illustrating an example of an operation performed by the gas analyzing system according to Embodiment.

FIG. 5 is a diagram illustrating an example of a signal outputted from a sensor according to Embodiment.

FIG. 6 is a diagram illustrating selection example 1 of extraction sections selected in a selecting step according to Embodiment.

FIG. 7 is a diagram illustrating selection example 2 of extraction sections selected in the selecting step according to Embodiment.

FIG. 8 is a diagram illustrating an example of division of a second period and a third period.

FIG. 9 is a flowchart illustrating another example of the operation performed by the gas analyzing system according to Embodiment.

### DESCRIPTION OF EMBODIMENT

### Circumstances Leading to Aspect of the Present Disclosure

For an analysis of molecules contained in a gas using a sensor that outputs a signal responsive to molecular adsorption, qualitative analysis or quantitative analysis is performed based on features of a signal outputted from the sensor that is exposed to a sample gas containing an analysis target molecule, for example. Molecular behavior of adsorption to or desorption from the sensor changes according to, for example, the kind of molecule contained in the sample gas and the concentration of molecule in the sample gas. Thus, the signal outputted from the sensor fluctuates. With this, the molecules contained in the sample gas are analyzed based on features of the signal outputted from the sensor in a period during which the sensor is exposed to the sample gas. Examples of the features include a signal value, the amount of change in the signal value, and a change rate of the signal value. However, if the sample gas contains different kinds of molecules, the signal outputted from the sensor also fluctuates depending on the presence or absence of, or a change in the amount of, molecules other than the analysis target molecule. This results in a decrease in the accuracy in analyzing the analysis target molecule. Thus, gas analysis methods capable of increasing the analytical accuracy even in the presence of a molecule other than the analysis target molecule have been awaited.

The inventors found out that even if the sample gas contained different kinds of molecules, the fluctuations of the signal outputted from the sensor were small immediately after exposing the sensor to the sample gas or immediately after stopping the sensor from being exposed after exposing the sensor to the sample gas. More specifically, there are a period where the effect of the different kinds of molecules in the sample gas on the signal is large and a period where the effect is small. On this account, an effective way is to use a feature of the obtained signal outputted from the sensor in a partial period, depending on the analytical purpose. Furthermore, depending on an adsorbing material included in the sensor, the signal outputted from the sensor changes. For this reason, another effective way is to use features of signals outputted from a plurality of sensors including mutually different adsorbing materials. In view of these findings, the present disclosure has an object to provide a gas analyzing method that is capable of increasing accuracy in analyzing molecules contained in a gas.

### Summary of Present Disclosure

The following is a summary according to an aspect of the present disclosure.

In accordance with an aspect of the present disclosure, a gas analyzing method using a sensor that outputs a signal responsive to molecular adsorption includes: obtaining a signal outputted from the sensor that is exposed to a sample gas in a measurement period including a first period, a second period following the first period, and a third period following the second period, under a condition where a molecule contained in the sample gas is more readily adsorbed to the sensor in the second period than in the first period and the third period; dividing at least one of the second period or the third period into a plurality of divided sections, and selecting, as at least one extraction section, a part of the plurality of divided sections; extracting at least one feature of the signal outputted in the at least one extraction section; and performing, using a pre-trained logical model for one of qualitative determination or quantitative determination of the molecule contained in the sample gas, one of qualitative analysis or quantitative analysis on the molecule contained in the sample gas, based on only the at least one feature extracted in the extracting among features of the signal in the measurement period, and outputting an analysis result of the performing of the one of qualitative analysis or quantitative analysis.

With this, the sample-gas analysis is performed based on, among the features in the measurement period, only the at least one feature of the signal in the at least one extraction section included in the second and third periods. The measurement period includes a period where the signal value outputted from the sensor is more likely to be affected by the kind of molecule contained in the sample gas and a period where the signal value is less likely to be affected by the kind of molecule contained in the sample gas. On this account, even if the sample gas contains a different molecule other than the analysis target molecule, the selecting enables the at least one extraction section, in which the effect of this different molecule is small, to be selected. Thus, according to the present aspect, selecting the at least one extraction section depending on the analytical purpose increases the accuracy in analyzing the molecules contained in the gas even if the gas contains a different molecule other than the analysis target molecule.

For example, it is possible that in the selecting, the at least one extraction section is selected from among a divided section counted second from a beginning of each of the second period and the third period and one or more divided sections subsequent to the divided section in each of the second period and the third period among the plurality of divided sections.

With the passage of time after each beginning of the second period and the third period, the kind of molecule is more likely to affect the output of the sensor. For this reason, the at least one extraction section is selected from the second and subsequent divided sections counted from the beginning in each of the second period and the third period. This enables the sample-gas analysis to be performed based on the feature of the signal in the section where the kind of molecule has a large effect. Hence, the analytical accuracy in identifying the kind of molecule can be increased.

For example, it is possible that in the selecting, the at least one extraction section is selected from a last divided section in each of the second period and the third period among the plurality of divided sections.

With this, the sample-gas analysis can be performed based on the feature of the signal in the section where the kind of molecule has a particularly large effect. Hence, the analytical accuracy in identifying the kind of molecule can be increased.

For example, it is possible that in the selecting, the at least one extraction section is selected from divided sections in the third period among the plurality of divided sections.

In the third period, the signal indicating that the molecules contained in the sample gas are desorbed from the sensor is outputted from the sensor. Desorption of the molecules from the sensor is affected by the adsorbed state of the molecules. On this account, the signal outputted from the sensor is particularly likely to show the effect of the kind of molecule. Thus, from among the plurality of divided sections, the at least one extraction section is to be selected from the divided sections of the third period. This further increases the analytical accuracy in identifying the kind of molecule.

For example, it is possible that in the performing of the one of qualitative analysis or quantitative analysis, analysis to identify a kind of the molecule contained in the sample gas is performed.

With this, the kind of molecule contained in the sample gas can be identified with high accuracy.

For example, it is possible that in the performing of the one of qualitative analysis or quantitative analysis, analysis to identify a kind of an organic compound as the molecule contained in the sample gas is performed.

With this, the kind of organic compound contained in the sample gas can be identified with high accuracy.

For example, it is possible that in the selecting, the at least one extraction section is selected from a divided section counted second from an end of each of the second period and the third period and one or more divided sections preceding the divided section in each of the second period and the third period among the plurality of divided sections.

With this, even if the sample gas contains a different molecule other than the predetermined molecule, the sample-gas analysis can be performed based on the feature of the signal in the section where this different molecule has a small effect. Hence, the analytical accuracy in determining the concentration of the predetermined molecule can be increased.

For example, it is possible that in the selecting, the at least one extraction section is selected from a divided section counted first in each of the second period and the third period among the plurality of divided sections.

With this, even if the sample gas contains a different molecule other than the predetermined molecule, the sample-gas analysis can be performed based on the feature of the signal in the section where this different molecule has a particularly small effect. Hence, the analytical accuracy in determining the concentration of the predetermined molecule can be further increased.

For example, it is possible that in the performing of the one of qualitative analysis or quantitative analysis, analysis to determine a concentration of a predetermined molecule contained in the sample gas is performed.

With this, the concentration of the predetermined molecule contained in the sample gas can be determined with high accuracy.

For example, it is possible that in the performing of the one of qualitative analysis or quantitative analysis, analysis to determine a concentration of a water molecule as the predetermined molecule contained in the sample gas is performed.

With this, the concentration of water molecule contained in the sample gas can be determined with high accuracy.

For example, it is possible that the at least one feature includes at least one of: a difference between a value of the signal after change due to exposure of the sensor to the sample gas and a value of the signal before the change; or an amount of change in the value of the signal per unit time.

With this, the at least one feature that is likely to reflect the effect of the molecular adsorption to the sensor is used.

For example, it is possible that in the obtaining, the signal outputted from the sensor exposed to the sample gas only in the second period of the measurement period is obtained.

With this, because the sensor is not exposed to the sample gas in the first period and the third period, the signal value outputted from the sensor in the measurement period significantly fluctuates. This increases the analytical accuracy.

In accordance with another aspect of the present disclosure, a gas analyzing method using a plurality of sensors each of which outputs a signal responsive to molecular adsorption, the plurality of sensors exhibiting mutually different molecular adsorption behaviors includes: obtaining a plurality of signals outputted from each of the plurality of sensors that are exposed to a sample gas in a measurement period including a first period, a second period following the first period, and a third period following the second period, under a condition where a molecule contained in the sample gas is more readily adsorbed to the plurality of sensors in the second period than in the first period and the third period; extracting a plurality of features from each of the plurality of signals; and performing analysis to determine, using a pre-trained logical model for determining a concentration of a predetermined molecule contained in the sample gas, the concentration of the predetermined molecule contained in the sample gas, based on the plurality of features extracted in the extracting, and outputting an analysis result of the performing of analysis.

With this, the concentration of the predetermined molecule contained in the sample gas is determined based on the plurality of features extracted from the signals outputted from the plurality of sensors which exhibit mutually different molecular adsorption behaviors. Thus, even if the sample gas contains a molecule other than the analysis target molecule, the use of the plurality of features extracted as described reduces the effect of this different molecule on the analysis result. Hence, according to the present aspect, the analytical accuracy can be increased even in the presence of the different molecule other than the analysis target molecule.

For example, it is possible that in the performing of analysis, a concentration of a water molecule as the predetermined molecule contained in the sample gas is determined.

With this, the concentration of water molecule contained in the sample gas can be determined with high accuracy.

For example, it is possible that in the obtaining, the signal outputted from the sensor is obtained via a network.

With this, the signal from the sensor measured at a different location can be obtained.

For example, it is possible that the sensor includes a sensitive membrane.

For example, it is possible that the sensitive membrane includes a resin material and conductive particles dispersed in the resin material.

With this, the accuracy in analyzing the molecules contained in the gas can be increased even if the sensor is a sensitive membrane that is likely to be affected by the presence of a different molecule other than the analysis target molecule in the sample gas.

In accordance with still another aspect of the present disclosure, a gas analyzing system includes: a sensor that outputs a signal responsive to molecular adsorption; an exposer that exposes the sensor to a sample gas in a measurement period including a first period, a second period following the first period, and a third period following the second period, under a condition where a molecule contained in the sample gas is more readily adsorbed to the sensor in the second period than in the first period and the third period; an obtainer that obtains the signal outputted from the sensor in the measurement period; a selector that divides at least one of the second period or the third period into a plurality of divided sections, and selects, as at least one extraction section, a part of the plurality of divided sections; an extractor that extracts at least one feature of the signal outputted in the at least one extraction section; a memory that stores a pre-trained logical model for one of qualitative determination or quantitative determination of the molecule contained in the sample gas; and an analyzer that performs, using the pre-trained logical model, one of qualitative analysis or quantitative analysis on the molecules contained in the sample gas, based on only the at least one feature extracted by the extractor among features of the signal in the measurement period, and outputs an analysis result of the one of qualitative analysis or quantitative analysis.

With this, the sample-gas analysis is performed based on, among the features in the measurement period, only the at least one feature of the signal in the at least one extraction section included in the second and third periods. The measurement period includes a period where the signal value outputted from the sensor is more likely to be affected by the kind of molecule contained in the sample gas and a period where the signal value is less likely to be affected by the kind of molecule contained in the sample gas. On this account, even if the sample gas contains a different molecule other than the analysis target molecule, the selector is able to select the at least one extraction section in which the effect of this different molecule is small. Thus, according to the present aspect, selecting the at least one extraction section depending on the analytical purpose increases the accuracy in analyzing the molecules contained in the gas even if the gas contains a different molecule other than the analysis target molecule.

In accordance with still another aspect of the present disclosure, a gas analyzing system includes: a plurality of sensors each of which outputs a signal responsive to molecular adsorption, the plurality of sensors exhibiting mutually different molecular adsorption behaviors; an exposer that exposes the plurality of sensors to a sample gas in a measurement period including a first period, a second period following the first period, and a third period following the second period, under a condition where a molecule contained in the sample gas is more readily adsorbed to the plurality of sensors in the second period than in the first period and the third period; an obtainer that obtains a plurality of signals outputted from each of the plurality of sensors in the measurement period; an extractor that extracts a plurality of features from each of the plurality of signals; a memory that stores a pre-trained logical model for determining a concentration of a predetermined molecule contained in the sample gas; and an analyzer that performs analysis to determine, using the pre-trained logical model, the concentration of the predetermined molecule contained in the sample gas, based on the plurality of features extracted by the extractor, and outputs an analysis result of the analysis.

With this, the concentration of the predetermined molecule contained in the sample gas is determined based on the plurality of features extracted from the signals outputted from the plurality of sensors each which exhibit mutually different molecular adsorption behaviors. Thus, even if the sample gas contains a molecule other than the analysis target molecule, the use of the plurality of features extracted as described reduces the effect of this different molecule on the analysis result. Hence, according to the present aspect, the analytical accuracy can be increased even in the presence of the different molecule other than the analysis target molecule.

Hereinafter, certain exemplary embodiments will be described in detail with reference to the accompanying Drawings. The following embodiments are specific examples of the present disclosure. The numerical values, shapes, materials, elements, arrangement and connection configuration of the elements, steps, the order of the steps, etc., described in the following embodiments are merely examples, and are not intended to limit the present disclosure. Among elements in the following embodiments, those not described in any one of the independent claims indicating the broadest concept of the present disclosure are described as optional elements.

It should also be noted that the following description may include terms indicating relationships between structural elements, such as “parallel”, terms indicating shapes of structural elements, and numerical value ranges. However, they do not mean exact meanings only. They also mean the substantially same ranges including a difference of, for example, about several % from the completely same range.

It should also be noted that the respective figures are schematic diagrams and are not necessarily precise illustrations. Additionally, components that are essentially the same share like reference signs in the figures. Accordingly, overlapping explanations thereof are omitted or simplified.

### Embodiment

### Configuration

The configuration of a gas analyzing system according to Embodiment is firstly described.

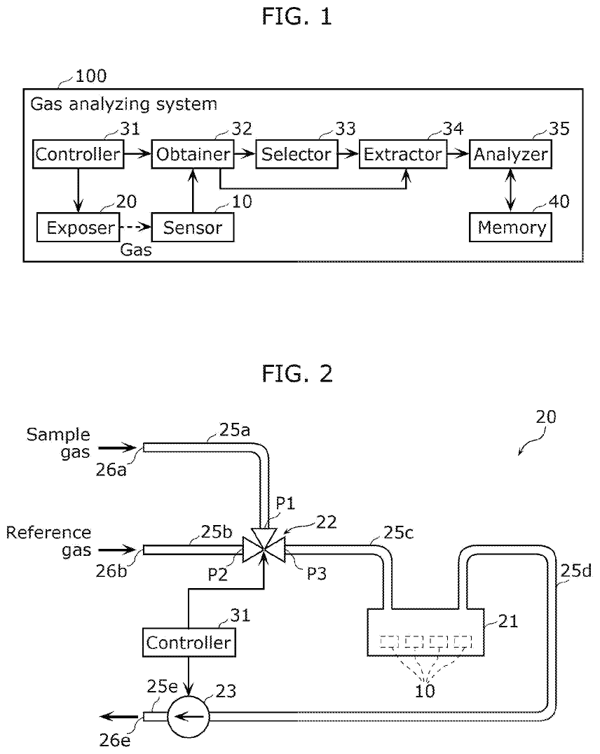

FIG. 1 is a schematic block diagram illustrating the configuration of gas analyzing system 100 according to the present embodiment.

As illustrated in FIG. 1, gas analyzing system 100 according to the present embodiment includes sensor 10, exposer 20, controller 31, obtainer 32, selector 33, extractor 34, analyzer 35, and memory 40. Based on an output from sensor 10 exposed to a sample gas, gas analyzing system 100 analyzes the sample gas. To be more specific, gas analyzing system 100 performs qualitative analysis or quantitative analysis on molecules contained in the sample gas. The sample gas contains an analysis target molecule, such as a volatilized organic compound or water molecule. Examples of the sample gas include gas collected from food, collected exhaled human breath, air surrounding a human body, and air collected from a room in a building.

Gas analyzing system 100 performs qualitative analysis by identifying a kind of molecule contained in the sample gas, for example.

Any molecule that is adsorbed to sensor 10 may be the target molecule for the qualitative analysis. For example, the target molecule is an organic compound. The target molecule for the qualitative analysis may be an inorganic gas molecule, such as ammonia or carbon monoxide. Gas analyzing system 100 may be used for identifying an odor, for example. In this case, the organic compound is a molecule of an odor component.

Gas analyzing system 100 performs quantitative analysis by determining the concentration of a predetermined molecule contained in the sample gas, for example.

Any molecule that is adsorbed to sensor 10 may be a predetermined target molecule for the quantitative analysis. For example, the predetermined target molecule is a water molecule. More specifically, gas analyzing system 100 determines the humidity of the sample gas. The predetermined target molecule may be an organic compound or an inorganic gas molecule, such as ammonia or carbon monoxide.

Even for the sample gas containing a different molecule other than the analysis target molecule, gas analyzing system 100 is capable of increasing the analytical accuracy. The sample gas to be analyzed by gas analyzing system 100 contains an organic compound and a water molecule, for example. Even for the sample gas containing an organic compound and a water molecule, gas analyzing system 100 is capable of performing the analysis with high accuracy.

Sensor 10 outputs a signal responsive to molecular adsorption to sensor 10. More specifically, the signal outputted from sensor 10 changes depending on the molecular adsorption concentration. If the kind of molecule adsorbed to sensor 10 changes, the output signal also changes even if the molecular adsorption concentration is unchanged. For example, sensor 10 is an electrochemical sensor, a semiconductor sensor, a field-effect transistor sensor, a surface acoustic wave sensor, a quartz resonator sensor, or a resistance change sensor.

Sensor 10 includes a sensing part and a pair of electrodes electrically connected to the sensing part, for example. The sensing part is a sensitive membrane that changes in electrical resistance according to the molecular adsorption concentration, for example. The signal responsive to the electrical resistance of the sensing part of sensor 10 is obtained as a voltage signal or a current signal by obtainer 32 via the pair of electrodes.

For example, the sensing part is a sensitive membrane contains: a resin material as an adsorbent for adsorbing a target molecule for the analysis performed by gas analyzing system 100; and conductive particles dispersed in the resin material. Examples of the resin material include polyalkylene glycol resin, polyester resin, and silicon resin. The resin material is commercially available as a stationary phase for a gas chromatography column as a side chain. In terms of durability and molecular adsorptive property, the resin material may be a silicon resin containing substituent groups, such as a phenyl group and a methyl group, as side chains. Although the sensing part contains the resin material and the conductive particles, this is not intended to be limiting. The sensing part may be any structural member that changes in electrical resistance according to the adsorption of the analysis target molecule. The sensing part may contain inorganic material, such as metal oxide, or contain cellular ceramics.

Gas analyzing system 100 includes a plurality of sensors 10, for example. At least two of the plurality of sensors 10 include the sensing parts (or more specifically, the resin materials forming the sensing parts) that are made from different kinds of material, for example. Here, the different kinds of material used for the resin materials have different composition formulas, for example. The materials forming the sensing parts of the plurality of sensors 10 may be all different from each other. The different kinds of material exhibit different adsorption behaviors to the same kind of molecule. More specifically, each of the plurality of sensors 10 exhibits a different molecular adsorption behavior, for example. On this account, each of the plurality of sensors 10 outputs a different signal in response to adsorption of the same kind of molecule. This allows different features to be extracted from the outputs from the plurality of sensors 10. Hence, the analytical accuracy of gas analyzing system 100 can be increased.

Exposer 20 is an exposure mechanism that exposes sensor 10 to a gas under the control of controller 31 in a measurement period including a first period, a second period following the first period, and a third period following the second period. To be more specific, exposer 20 exposes sensor 10 to a sample gas under a condition where the analysis target molecule contained in the sample gas is more readily adsorbed to sensor 10 in the second period than in the first period and the third period of the measurement period. For example, exposer 20 exposes sensor 10 to the sample gas only in the second period of the measurement period. This allows the concentration of the analysis target molecule around sensor 10 in the second period to be higher than in the first period and the third period. As a result, the analysis target molecule is more readily adsorbed to sensor 10 in the second period. Furthermore, because sensor 10 is not exposed to the sample gas in the first period and the third period, the signal value outputted from sensor 10 significantly fluctuates in the measurement period. This increases the analytical accuracy.

Exposer 20 may expose sensor 10 to a reference gas in the first period and the third period. The reference gas, which is a measurement reference gas, is different from the sample gas in composition. For example, the reference gas contains no analysis target molecule or contains the analysis target molecule with a significantly lower concentration than the concentration in the sample gas (one-tenth or lower, for instance). The composition of the reference gas remains the same for each measurement. The reference gas contains a molecule less likely to be adsorbed to the sensing part of sensor 10 than the analysis target molecule.

Specific examples of the reference gas include: air for industrial or analytical use; an inert gas, such as nitrogen or noble gas, that contains substantially no water molecules nor organic compounds; and a gas obtained by removing the analysis target molecules from the sample gas using a filter or the like. The exposure to the reference gas in the first period and the third period as described allows the signal from sensor 10 to be stable for each measurement even if the surrounding environment of sensor 10 changes. This increases the analytical accuracy described later.

The present embodiment below describes mainly the case where exposer 20 exposes sensor 10 to the sample gas in the second period and to the reference gas in the first period and the third period.

Here, the configuration of exposer 20 is described in detail. FIG. 2 is a schematic diagram illustrating an example of the configuration of exposer 20 according to the present embodiment. As illustrated in FIG. 2, exposer 20 includes casing 21, three-way solenoid valve 22, suction pump 23, and a plurality of pipes 25a, 25b, 25c, 25d, and 25e.

At one end of pipe 25a, inlet 26a is provided to introduce the sample gas. Inlet 26a is provided in a space filled with the sample gas, for example. At one end of pipe 25b, inlet 26b is provided to introduce the reference gas. Inlet 26b is provided in a space filled with the reference gas, for example. At one end of pipe 25e, outlet 26e is provided to discharge the introduced sample gas or the introduced reference gas.

Casing 21 is a box-shaped container that encases the plurality of sensors 10. The plurality of sensors 10 are arranged in an array in casing 21, for example. Casing 21 is connected to one end of pipe 25c and to one end of pipe 25d. Operation of suction pump 23, described later, allows the gas to flow from the one end of pipe 25c to the one end of pipe 25d. The plurality of sensors 10 are located in a flow path of the gas.

The sample gas introduced from inlet 26a is introduced into casing 21 via pipe 25a, three-way solenoid valve 22, and pipe 25c. The reference gas introduced from inlet 26b is introduced into casing 21 via pipe 25b, three-way solenoid valve 22, and pipe 25c. The sample gas and the reference gas introduced into casing 21 are discharged from outlet 26e via pipe 25d, suction pump 23, and pipe 25e.

Three-way solenoid valve 22 is a solenoid valve for switching the gas to be introduced into casing 21. Three-way solenoid valve 22 includes: input port P1 connected to the other end of pipe 25a; input port P2 connected to the other end of pipe 25b; and output port P3 connected to the other end of pipe 25c. The opening and closing of these ports of three-way solenoid valve 22 are controlled by controller 31. Under the control of controller 31, three-way solenoid valve 22 switches between a first state in which input port P1 and output port P3 are electrically connected and a second state in which input port P2 and output port P3 are electrically connected. In the first state, input port P1 and output port P3 are open and input port P2 is closed. In the second state, input port P2 and output port P3 are open and input port P1 is closed.

Suction pump 23 introduces the sample gas and the reference gas into casing 21 and discharges the introduced sample gas and the introduced reference gas from outlet 26e. The operation of suction pump 23 is controlled by controller 31. An inlet of suction pump 23 is connected to the other end of pipe 25d. An outlet of suction pump 23 is connected to the other end of pipe 25e.

This configuration enables the sample gas to be introduced into casing 21 in the first state of three-way solenoid valve 22 while suction pump 23 is operating. Thus, exposer 20 exposes the plurality of sensors 10 to the sample gas. In the second state of three-way solenoid valve 22 while suction pump 23 is operating, the reference gas is introduced into casing 21. Thus, exposer 20 exposes the plurality of sensors 10 to the reference gas. Under such control performed on three-way solenoid valve 22, the plurality of sensors 10 are exposed only to the sample gas in the first state of three-way solenoid valve 22 and only to the reference gas in the second state of three-way solenoid valve 22.

Note that the configuration of exposer 20 is not limited to the one illustrated in FIG. 2. Exposer 20 may have any configuration that allows sensors 10 to be exposed to the sample gas. Exposer 20 may cause the sample gas and the reference gas to be introduced into casing 21 via a different pipe without passing through three-way solenoid valve 22. Alternatively, exposer 20 need not include suction pump 23 and may cause a carrier gas to constantly flow into casing 21 so that the sample gas is mixed into the carrier gas. Alternatively, the reference gas need not be introduced. In this case, after sensors 10 are exposed to the sample gas, suction pump 23 may create a vacuum in casing 21. Alternatively, exposer 20 may include a temperature controller that controls the temperature inside casing 21. In this case, exposer 20 may expose sensors 10 to the sample gas in the whole measurement period under a condition where, by making the temperature in casing 21 in the second period lower than the temperatures in the first period and the third period, the analysis target molecules contained in the sample gas are more readily adsorbed to sensors 10 in the second period than in the first period and the third period. Moreover, exposer 20 may further include: removal filters for removing moisture and microparticles from the sample gas and the reference gas; a solenoid control valve that controls the flows of the pipes; and a check valve that prevents backflow of the pipes.

Referring back to FIG. 1, controller 31 controls the operation of exposer 20, or more specifically, the operations of three-way solenoid valve 22 and suction pump 23 illustrated in FIG. 2, as described above. Controller 31 may output, to obtainer 32, information indicating operation timings of exposer 20 (for example, information on times and period lengths of the first, second, and third periods).

Obtainer 32 obtains the signal outputted from sensor 10 in the measurement period. For example, obtainer 32 obtains a voltage signal or a current signal, as the signal responsive to the electrical resistance of the sensing part of sensor 10.

Selector 33 divides at least one of the second period and the third period of the signal obtained by obtainer 32 into at least two sections, for example. Then, from a part of a plurality of divided sections as a result of the division, selector 33 selects at least one extraction section.

Extractor 34 extracts a feature of the signal obtained by obtainer 32. When more than one sensor 10 is included, extractor 34 extracts a feature for each of the signals outputted from the plurality of sensors 10.

Using a pre-trained logical model for example, analyzer 35 performs qualitative analysis or quantitative analysis on molecules contained in the sample gas, based on the feature extracted by extractor 34. Analyzer 35 performs qualitative analysis by identifying the kind of molecule contained in the sample gas, for example. Analyzer 35 performs quantitative analysis by determining the concentration of a predetermined molecule contained in the sample gas, for example.

Analyzer 35 receives an input of the feature extracted by extractor 34 and then outputs an analysis result. For example, analyzer 35 outputs information used for displaying the analysis result on, for example, a display (not shown) provided for the gas analyzing system. Analyzer 35 may output the information indicating the analysis result to memory 40 and cause memory 40 to store this information. Furthermore, analyzer 35 may output the information indicating the analysis result to an external device.

Controller 31, obtainer 32, selector 33, extractor 34, and analyzer 35 are implemented by a microcomputer or a processor having built-in programs to perform the aforementioned and after-mentioned processes. Controller 31, obtainer 32, selector 33, extractor 34, and analyzer 35 each may be implemented by a dedicated logic circuit that performs the aforementioned and after-mentioned processes.

Memory 40 is a storage device that stores the pre-trained logical model used by analyzer 35. Memory 40 is implemented by a semiconductor memory, for example.

The pre-trained logical model is a logical model that performs qualitative determination or quantitative determination on the molecules contained in the sample gas. The pre-trained logical model receives an input of the feature extracted by extractor 34 and then outputs a result of the qualitative analysis or quantitative analysis of the molecules contained in the sample gas.

The pre-trained logical model for qualitative determination of the molecules contained in the sample gas is a logical model that identifies the kind of molecule contained in the sample gas, for example. To be more specific, the pre-trained logical model is a logical model that identifies which one of a plurality of identification target molecules is contained in the sample gas. In this case, the pre-trained logical model receives an input of the feature extracted by extractor 34 and then outputs a result indicating which one of the plurality of identification target molecules is contained in the sample gas. The pre-trained logical model may receive an input of the feature extracted by extractor 34 and then output a result indicating whether any identification target molecule is contained in the sample gas.

The pre-trained logical model for quantitative determination of the molecules contained in the sample gas is a logical model that determines the concentration of the predetermined molecule contained in the sample gas, for example. To be more specific, the pre-trained logical model is a logical model that determines which one of a plurality of concentration candidates corresponds to the concentration of the predetermined molecule contained in the sample gas. In this case, the pre-trained logical model receives an input of the feature extracted by extractor 34 and then outputs a result indicating which one of the plurality of concentration candidates corresponds to the concentration of the predetermined molecule contained in the sample gas. The pre-trained logical model may receive an input of the feature extracted by extractor 34 and then output a result indicating the concentration of the predetermined molecule contained in the sample gas.

For example, the pre-trained logical model is built through machine learning using training data that includes: a known qualitative-analysis result or a known quantitative-analysis result; and a feature to be extracted by extractor 34 when the sample gas that provides the known qualitative-analysis result or the known quantitative-analysis result is used. The known qualitative-analysis result used as the training data indicates a kind of the identification target molecule, for example. The known quantitative-analysis result indicates a concentration of the predetermined molecule, for example.

The logical model in machine learning may be built according to any method. Examples of the method of building the logical model in machine learning include random forest, neural network, support vector machine, and self-organizing map. Thus, the pre-trained logical model includes at least one of random forest, neural network, support vector machine, and self-organizing map, for example.

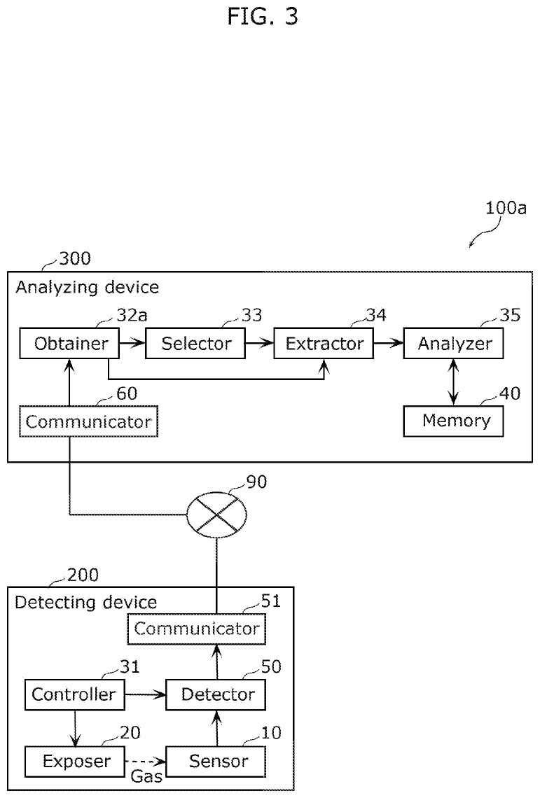

Gas analyzing system 100 is implemented as a single gas analyzing device including the aforementioned structural components, for example. However, gas analyzing system 100 may be implemented by a plurality of devices. If gas analyzing system 100 is implemented by a plurality of devices, the structural components included in gas analyzing system 100 may be freely dispersed in the plurality of devices. Here, an example of the gas analyzing system that is implemented by the plurality of devices is described with reference to FIG. 3. FIG. 3 is a schematic block diagram illustrating a configuration of gas analyzing system 100a according to a variation of Embodiment.

As illustrated in FIG. 3, gas analyzing system 100a includes detecting device 200 and analyzing device 300.

Detecting device 200 includes sensor 10, exposer 20, controller 31, detector 50, and communicator 51. Sensor 10, exposer 20, and controller 31 are the same as those included in gas analyzing system 100 described above, for example.

Detector 50 obtains a signal outputted from sensor 10 in a measurement period. For example, detector 50 obtains a voltage signal or a current signal as a signal responsive to the electrical resistance of a sensing part of sensor 10. Moreover, information indicating operation timings of exposer 20 may be obtained from controller 31. Detector 50 transmits the signal and information obtained, to analyzing device 300 via communicator 51. Detector 50 is implemented by a microcomputer or a processor having a built-in program to perform the aforementioned process. Detector 50 may be implemented by a dedicated logic circuit that performs the aforementioned process.

Communicator 51 is a communication module (a communication circuit) that enables detecting device 200 to communicate with analyzing device 300 via wide area communication network 90, which is an example of a network, such as the Internet. Communicator 51 may perform wired communication or wireless communication. Communicator 51 may perform communication according to any communication standard.

Analyzing device 300 includes obtainer 32a, selector 33, extractor 34, analyzer 35, memory 40, and communicator 60. Selector 33, extractor 34, analyzer 35, and memory 40 are the same as those included in gas analyzing system 100 described above.

Obtainer 32a obtains, via wide area communication network 90, the signal that is outputted from sensor 10 in the measurement period and obtained by detector 50. Using communicator 60, obtainer 32a communicates with detecting device 200 via wide area communication network 90. Extractor 34 extracts a feature of the signal obtained by obtainer 32a.

Communicator 60 is a communication module (a communication circuit) that enables analyzing device 300 to communicate with detecting device 200 via wide area communication network 90. Communicator 60 may perform wired communication or wireless communication. Communicator 60 may perform communication according to any communication standard.

### Operation

Next, the operation of the gas analyzing system according to the present embodiment is described. Although the following mainly describes the operation of gas analyzing system 100, gas analyzing system 100a also performs the same operation unless otherwise noted.

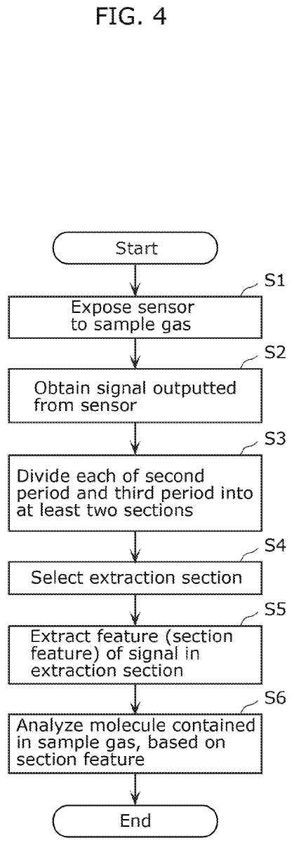

FIG. 4 is a flowchart illustrating an example of the operation performed by gas analyzing system 100 according to the present embodiment. To be more specific, FIG. 4 is a flowchart of the gas analyzing method used by gas analyzing system 100. The gas analyzing method according to the present embodiment includes an exposing step, an obtaining step, a selecting step, an extracting step, and an analyzing step, for example.

### (1) Exposing Step and Obtaining Step

As illustrated in FIG. 4, exposer 20 exposes sensor 10 to the sample gas only in the second period of the measurement period in the exposing step (Step S1). In this way, exposer 20 exposes sensor 10 to the sample gas under the condition where the molecules contained in the sample gas are more readily adsorbed to sensor 10 in the second period than in the first period and the third period of the measurement period in which sensor 10 is exposed to the sample gas. Furthermore, exposer 20 exposes sensor 10 to the reference gas in the first period and the third period in the exposing step. For example, controller 31 causes suction pump 23 to operate and controls the opening and closing of the ports of three-way solenoid valve 22. This allows sensor 10 to be exposed to the reference gas in the first and third periods and to the sample gas in the second period. Controller 31 causes exposer 20 to perform these processes, for example.

In the obtaining step, obtainer 32 obtains the signal outputted from sensor 10 exposed in Step S1 (Step S2). More specifically, obtainer 32 obtains the signal outputted from sensor 10 exposed to the sample gas under the condition where the molecules contained in the sample gas are more readily adsorbed to sensor 10 in the second period than in the first period and the third period. To be more specific, obtainer 32 obtains the signal outputted from sensor 10 exposed to the sample gas only in the second period of the measurement period.

Note that detector 50 of gas analyzing system 100a obtains the signal outputted from sensor 10 exposed to the sample gas in Step S1. Obtainer 32a obtains the signal outputted from sensor 10 exposed in Step S1, from detector 50 via wide area communication network 90. This allows obtainer 32a to obtain the signal outputted from sensor 10 even if sensor 10 is located away from analyzing device 300.

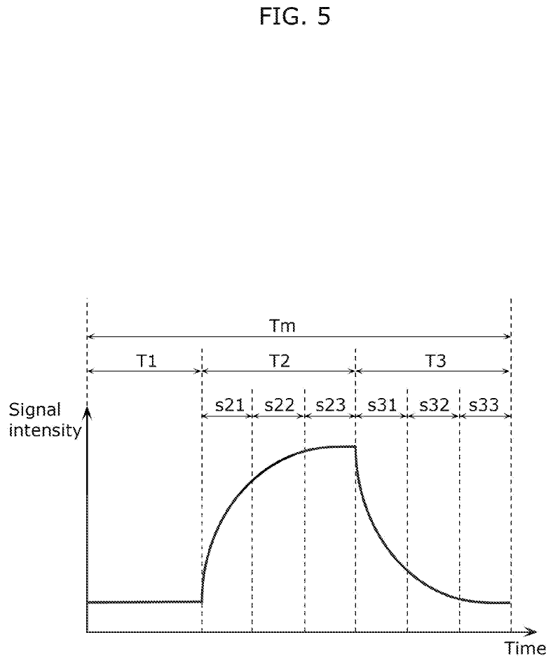

FIG. 5 is a diagram illustrating an example of the signal outputted from sensor 10. FIG. 5 illustrates an example of temporal change in the intensity (for example, the voltage) of the signal outputted from sensor 10 in measurement period Tm including first period T1, second period T2, and third period T3.

In Step S1, three-way solenoid valve 22 is in the second state in first period T1 and third period T3 of measurement period Tm and thus exposer 20 exposes sensor 10 to the reference gas, for example. Furthermore, three-way solenoid valve 22 is in the first state in second period T2 of measurement period Tm and thus exposer 20 exposes sensor 10 to the sample gas. More specifically, sensor 10 is exposed to the reference gas only in first period T1 and third period T3 of measurement period Tm, and to the sample gas only in second period T2 of measurement period Tm. As a result, the signal obtained in Step S2 changes as illustrated in FIG. 5, for example.

In first period T1 in which sensor 10 is exposed to the reference gas, the signal value shows little change. In subsequent second period T2 in which sensor 10 is exposed to the sample gas, the signal value changes (for example, increases) in response to adsorption of the molecules contained in the sample gas to the sensing part of sensor 10. Then, in third period T3 in which sensor 10 is exposed to the reference gas again, the signal value changed in the second period is returning to a reference value in response to desorption of the molecules having been adsorbed to the sensing part of sensor 10. The reference value is, for example, the value before the signal value starts changing as a result of the exposure of sensor 10 to the sample gas (or more specifically, the value immediately before the start of second period T2 or the value at the end of first period T1). In other words, the reference value is the value outputted from sensor 10 that is kept unexposed to the sample gas, or more specifically, kept exposed to the reference gas, for instance.

First period T1, second period T2, and third period T3 each have any time length. For example, the time lengths of these periods are set according to the analytical purpose, kind of sensor 10, or kind of the analysis target molecule, for example. The length of first period T1 is not less than 1 second and not more than 30 seconds, for example. The length of second period T2 is not less than 3 seconds and not more than 30 seconds, for example. The length of third period T3 is not less than 5 seconds and not more than 100 seconds, for example.

### (2) Selecting Step

Referring back to FIG. 4, selector 33 next performs the selecting step which includes: dividing second period T2 and third period T3 each into at least two sections (Step S3); and selecting at least one extraction section from a part of the plurality of divided sections resulting from the dividing in Step S3 (Step S4). The extraction section is used for extracting a feature of the signal in the extracting step.

In Step S3, second period T2 and third period T3 each are divided into the plurality of divided sections (for example, sections s21 to s23 and s31 to s33 as illustrated in FIG. 5). Note that, if selecting the at least one extraction section from the plurality of sections in either one of second period T2 and third period T3, selector 33 may divide only the corresponding one of second period T2 and third period T3 into at least two sections.

In Step S3, selector 33 divides second period T2 and third period T3 each into sections having the same length of time (that is, equal-time division). As a result of this, second period T2 is divided into the plurality of divided sections s21 to s23, and third period T3 is divided into the plurality of divided sections s31 to s33. Selector 33 may divide second period T2 and third period T3 each into any number of sections. The number of divided sections is set according to the analytical purpose, kind of sensor 10, or kind of the analysis target molecule, for example. To increase the analytical accuracy, the number of sections in each of second period T2 and third period T3 may be not less than two and not more than five, or not less than two and not more than three. Although the number of sections in second period T2 is equal to the number of sections in third period T3 in the example illustrated in FIG. 5, these numbers may be different.

Here, the selecting step, or more specifically, an example of selecting the extraction section in Step S4, is described.

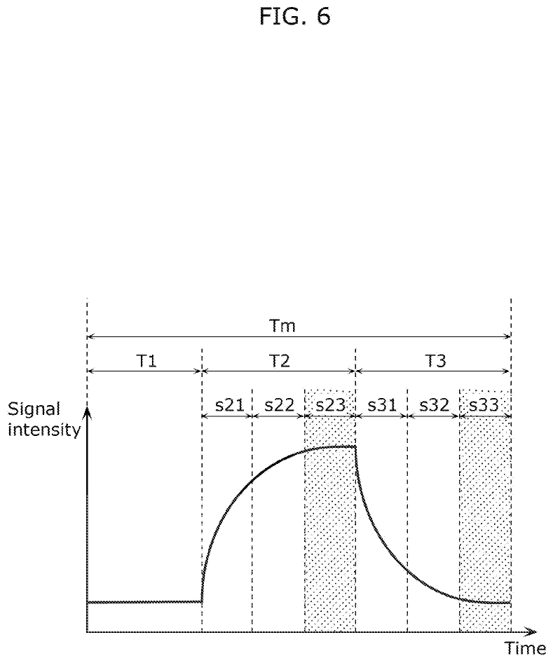

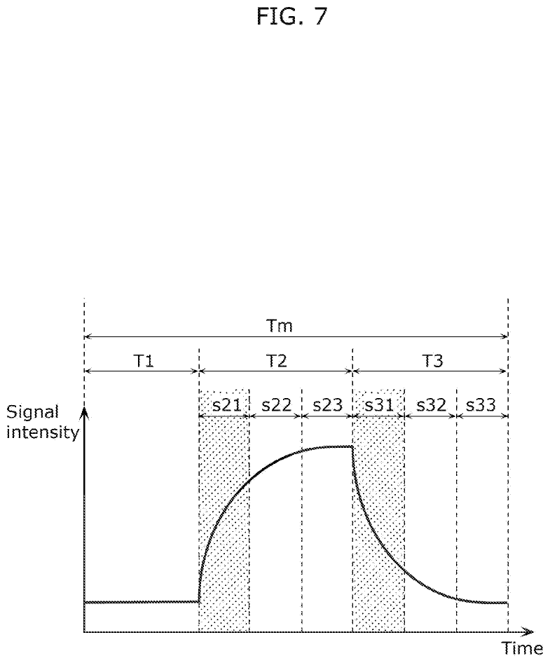

FIG. 6 is a diagram illustrating selection example 1 of the extraction sections selected in the selecting step. FIG. 7 is a diagram illustrating selection example 2 of extraction sections selected in the selecting step. In FIG. 6 and FIG. 7, the extraction sections selected from among the plurality of divided sections by selector 33 are represented by dot pattern.

Selection example 1 is first described. Selection example 1 corresponds to the selecting step that is performed when the kind of molecule contained in the sample gas is identified in the analyzing step, for example. As illustrated in FIG. 6, selector 33 in selection example 1 selects the at least one extraction section from the respective three sections in second period T2 and in third period T3. More specifically, selector 33 selects: section s23, which is the third from the beginning in second period T2; and section s33, which is the third from the beginning in third period T3. In this way, from among the plurality of divided sections, selector 33 selects the at least one extraction section from the second and subsequent divided sections from the beginning in each of second period T2 and third period T3, for example. Here, from among the plurality of divided sections, selector 33 may select, as the at least one extraction section, the last divided section in each of second period T2 and third period T3. Alternatively, from among the plurality of divided sections, selector 33 may select the at least one extraction section from the divided sections each of which occupies more than half of the latter half of the corresponding one of second period T2 and third period T3. In the present specification, a preceding divided section and a subsequent divided section are defined by a comparison of their beginning or ending times. More specifically, the divided section that begins or ends at an earlier time is defined as the preceding divided section.

Molecular adsorption or desorption easily takes place on the surface of the sensing part of sensor 10, regardless of the kind of molecule. On this account, the kind of molecule is less likely to affect the output of sensor 10 during a section in which molecular adsorption or desorption takes place mainly on the sensing part. More specifically, such a section is immediately after sensor 10 is exposed to the sample gas (that is, an earlier time in second period T2), or immediately after sensor 10 is stopped from being exposed to the sample gas (that is, an earlier time in third period T3). Meanwhile, the molecules adsorbed to the sensing part diffuse inside the sensing part. The speed of molecular diffusion inside the sensing part depends on the degree of interaction between the molecules and the sensing part, for example. With the passage of time after the beginning of second period T2 or third period T3, the kind of molecule is more likely to affect the output of sensor 10. For this reason, the extraction section is selected from among the second and subsequent divided sections from the beginning in each of second period T2 and third period T3. This enables the sample-gas analysis to be performed based on the feature of the signal outputted in the section where the kind of molecule has a large effect. As a result, the accuracy in identifying the kind of molecule can be increased.

For example, assume that the kind of organic compound that is an odor component is to be identified in the presence of water molecules. For example, the organic compound that is an odor component has a molecular weight of at least 40, which is relatively high, and thus the organic compounds that are odor components diffuse slowly. In contrast, the water molecule has a molecular weight of 18, which is relatively low, and thus the water molecules diffuse fast. On this account, the water molecules are more likely to affect the output of sensor 10 from each earlier time in second period T2 and third period T3. Hence, to identify the kind of organic compound that is an odor component in the presence of water molecules, the extraction section is to be selected from among the second and subsequent divided sections from the beginning in each of second period T2 and third period T3. This significantly increases the accuracy in identifying the kind of molecule.

Selector 33 may select, among the plurality of divided sections, the at least one extraction section only from the divided sections of second period T2 or only from among the divided sections of third period T3. For example, selector 33 selects only section s33 of third period T3 as the extraction section, from among the plurality of divided sections s21 to s23 and s31 to s33. In third period T3, the signal indicating that the molecules contained in the sample gas are desorbed from the sensing part of sensor 10 is outputted from sensor 10. Molecular desorption from the sensing part is also affected by the adsorbed state of the molecules. Thus, the signal outputted from sensor 10 is particularly likely to show the effect of the kind of molecule. On this account, the at least one extraction section is to be selected from the divided sections of third period T3, among the plurality of divided sections. This further increases the accuracy in identifying the kind of molecule.

Next, selection example 2 is described. Selection example 2 corresponds to the selecting step that is performed when the concentration of the predetermined molecule is determined in the analyzing step, for example. As illustrated in FIG. 7, selector 33 in selection example 2 selects the at least one extraction section from the respective three sections in second period T2 and in third period T3. More specifically, selector 33 selects: section s21, which is the first from the beginning in second period T2; and section s31, which is the first from the beginning in third period T3. In this way, from among the plurality of divided sections, selector 33 selects the at least one extraction section from the second and preceding divided sections from the end in each of second period T2 and third period T3, for example. Here, from among the plurality of divided sections, selector 33 may select, as the at least one extraction section, the first divided section that is at a beginning of each of second period T2 and third period T3. Alternatively, from among the plurality of divided sections, selector 33 may select the at least one extraction section from the divided sections each of which occupies more than half of the first half of the corresponding one of second period T2 and third period T3.

With the passage of time after the beginning of second period T2 or third period T3, the output of sensor 10 is more likely to be affected by the molecular diffusion inside the sensing part as described above. This indicates that the kind of molecule adsorbed to the sensing part has a significant effect. For this reason, the extraction section is selected from among the second and preceding divided sections from the end in each of second period T2 and third period T3. This enables, even if the sample gas contains a different kind of molecule other than the predetermined molecule, the sample-gas analysis to be performed based on the feature of the signal outputted in the section where this different kind of molecule has a relatively small effect. As a result, the accuracy in determining the concentration of the predetermined molecule camera can be increased.

For example, assume that the concentration of water molecules (that is, humidity) is to be determined in the presence of organic compounds that are odor components. As described above, the organic compounds that are odor components diffuse slowly inside the sensing part and, on the other hand, the water molecules diffuse fast inside the sensing part. On this account, the effect of the organic compounds that are odor components on the output of sensor 10 is likely to increase with the passage of time after each beginning of second period T2 and third period T3. Hence, to determine the humidity in the presence of the organic compounds that are odor components, the extraction section is to be selected from among the second and preceding divided sections from the end in each of second period T2 and third period T3. This significantly increases the accuracy in determining the humidity.

Note that although each of second period T2 and third period T3 is divided into the sections having the same length of time in the example illustrated in FIG. 5, this is not intended to be limiting. Selector 33 may divide each of second period T2 and third period T3 into sections having any time lengths. These time lengths are set according to the analytical purpose, kind of sensor 10, or kind of the analysis target molecule, for example.

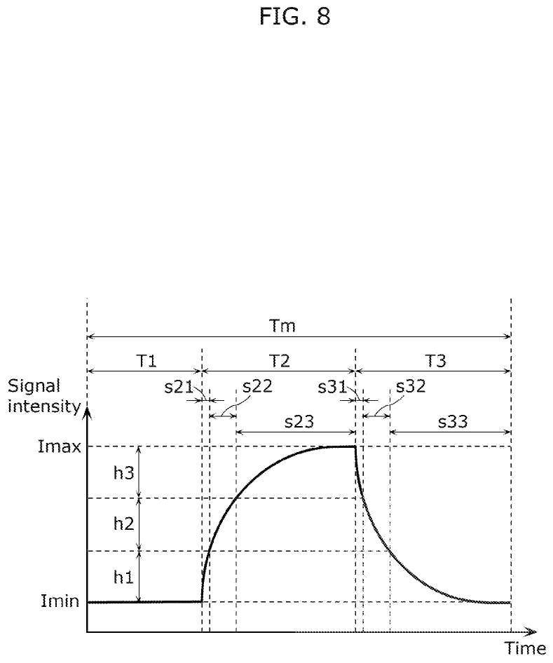

For example, selector 33 may divide the period so that the divided section where the obtained signal has a large amount of change in a signal value per unit time has a shorter length of time whereas the divided section where the obtained signal has a small amount of change in a signal value per unit time has a longer length of time. FIG. 8 is a diagram illustrating an example of division of second period T2 and third period T3.

FIG. 8 illustrates an example in which selector 33 divides second period T2 and third period T3 each into sections having an equal amount of change in signal value (that is, equal-wave-height division). To be more specific, selector 33 divides second period T2 and third period T3 each into sections by equally dividing a difference between minimum value Imin and maximum value Imax in each of second period T2 and third period T3, for example. For second period T2 and third period T3 each divided into three sections for instance, amounts of change h1 to h3 among sections s21 to s23 of second period T2 and among sections s31 to s33 of third period T3 are calculated by dividing the difference between minimum value Imin and maximum value Imax into equal thirds.

In this way, the equal-wave-height division is performed for each of second period T2 and third period T3 as described. As a result, the divided section where the obtained signal has a large amount of change in signal value per unit time has a shorter length of time whereas the divided section where the obtained signal has a small amount of change in signal value per unit time has a longer length of time. The amount of change in the signal value per unit time is used as an effective feature for analysis. Thus, dividing each of second period T2 and third period T3 to cause the value to change by a predetermined value or more increases the analytical accuracy. For example, selector 33 may select the at least one extraction section from the divided sections in each of which the amount of change in signal value changes by not less than one-fifth but not more than two-thirds of the difference between minimum value Imin and maximum value Imax. Alternatively, selector 33 may select the at least one extraction section from the divided sections in each of which the amount of change in signal value changes by not less than one-third but not more than two-thirds of the difference between minimum value Imin and maximum value Imax. Note that the signal value may change by any amount in the divided section. Selector 33 may divide each of second period T2 and third period T3 into sections each having a different amount of change in signal value.

Selector 33 may divide each of second period T2 and third period T3 into the at least one extraction section to be selected in Step S4 and a section that includes the other sections. For example, if section s33 is selected as the at least one extraction section in Step S4, selector 33 may divide third period T3 into: the section including sections s31 and s32; and section s33.

### (3) Extracting Step

Referring back to FIG. 4, in the extracting step, extractor 34 extracts at least one feature of the signal in the at least one extraction section selected in the selecting step (Step S5). Hereinafter, the at least one feature of the signal in the at least one extraction section that is extracted in the extracting step may also be referred to as a “section feature”.

As the section features of the signal in the at least one extraction section, extractor 34 extracts: a difference between a signal value after change due to the exposure of sensor 10 to the sample gas and a signal value before this change; and an amount of change in signal value per unit time (that is, the slope of the signal waveform), for example. This allows the analysis described later to be performed based on the at least one feature that is likely to reflect the effect of the molecular adsorption to the sensing part of sensor 10. Hence, the analytical accuracy can be increased. As the section feature, extractor 34 may extract either one of: the difference between the value after change due to the exposure of sensor 10 to the sample gas and the value before this change; and the amount of change in signal value per unit time. The value after the change due to the exposure of sensor 10 to the sample gas may be an average value of the signal values in the extraction section. Alternatively, the value after the change may be the signal value at a predetermined point in time in the extraction section (such as a point at the beginning, middle, or end of the extraction period). The value before the change may be the signal value at the end of first period T1, or may be an average value of the signal values in first period T1.

Note that extractor 34 may extract the section feature by any method that extracts the section feature without using the signal in any divided section other than the at least one extraction section among the plurality of divided sections. For example, extractor 34 may extract, as the section feature, each of signal values at predetermined intervals in the at least one extraction section. Alternatively, if deriving an approximate expression where the signal value is a time function, extractor 34 may extract a coefficient of the approximate expression as the section feature. Furthermore, extractor 34 extracts the section feature without converting the signal wave in a temporal dimension into a waveform in a different dimension, for example.

For gas analyzing system 100 including the plurality of sensors 10, Steps S2 to S5 are performed for each of the signals outputted from the plurality of sensors 10 so that the section feature is extracted for each of the signals outputted from the plurality of sensors 10.

### (4) Analyzing Step

In the analyzing step, using the pre-trained logical model for qualitative determination or quantitative determination of the molecules contained in the sample gas, analyzer 35 performs qualitative analysis or quantitative analysis on the molecules contained in the sample gas, based on only the section feature extracted by extractor 34 in the extracting step among the features of the signals in the measurement period, and outputs an analysis result (Step S6). Using the pre-trained logical model, analyzer 35 receives an input of the section feature extracted in the extracting step and outputs a result of the qualitative analysis or quantitative analysis performed on the molecules contained in the sample gas.

For an analysis by identifying the kind of molecule contained in the sample gas, analyzer 35 performs the analysis based on the section feature in the at least one extraction section described in selection example 1 above, for example. More specifically, analyzer 35 may identify the kind of organic compound as the molecule contained in the sample gas. For example, analyzer 35 outputs a result indicating which one of the plurality of identification target molecules is contained in the sample gas. Alternatively, analyzer 35 may identify whether an identification target molecule is contained in the sample gas. The identification-target organic compound has a molecular weight of not less than 40 and not more than 300 for example, and may have a molecule weight of not less than 100 and not more than 250. Although the organic compound having a molecular weight within such a range is adsorbed to the sensing part of sensor 10, the distribution speed inside the sensing part is relatively slow. This allows this organic compound to be easily identified with particularly high accuracy.

For an analysis by determining the concentration of the predetermined molecule contained in the sample gas, analyzer 35 performs the analysis based on the section feature in the at least one extraction section described in selection example 2 above, for example. More specifically, analyzer 35 may determine the concentration of water molecule as the predetermined molecule contained in the sample gas. For example, analyzer 35 outputs a result indicating which one of the plurality of concentration candidates corresponds to the concentration of the predetermined molecule contained in the sample gas.

As described above, the gas analyzing method is used by gas analyzing system 100 that includes sensor 10, for example. The gas analyzing method includes the obtaining step, the selecting step, the extracting step, and the analyzing step. In the obtaining step, the signal outputted from sensor 10 in measurement period Tm is obtained (Step S2). In the selecting step, at least one of second period T2 and third period T3 is divided into the plurality of divided sections (Step S3), and the at least one extraction section is selected from a part of the plurality of divided sections (Step S4). In the extracting step, the section feature of the signal in the at least one extraction section is extracted (Step S5). In the analyzing step, the sample-gas analysis is performed using the pre-trained logical model, based on only the section feature extracted in the extracting step from among the features of the signal in measurement period Tm, and an analysis result is outputted (Step S6).

In this way, the sample-gas analysis is performed based on only the feature (the section feature) of the signal in the at least one extraction section included in second period T2 and third period T3 among the features of the signals in measurement period Tm. Measurement period Tm includes a period where the signal value outputted from sensor 10 is more likely to be affected by the kind of molecule contained in the sample gas and a period where the signal value is less likely to be affected by the kind of molecule contained in the sample gas. On this account, even if the sample gas contains a different molecule other than the analysis target molecule, the selecting step enables the at least one extraction section, in which the effect of this different molecule is small, to be selected. As a result, selecting the at least one extraction section depending on the analytical purpose allows the section feature suitable for the analytical purpose to be extracted. Hence, the analytical accuracy can be increased even in the presence of the different molecule other than the analysis target molecule.

### (5) Another Example of Operation

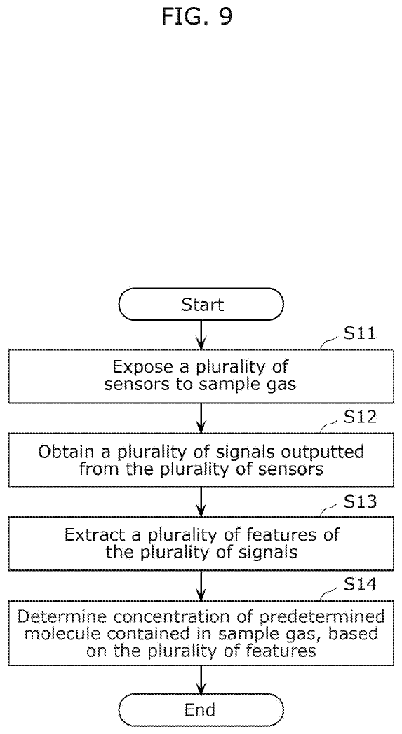

Next, another example of the operation performed by gas analyzing system 100 according to the present embodiment is described. FIG. 9 is a flowchart illustrating this other example of the operation performed by gas analyzing system 100 according to the present embodiment. This other example of the operation in FIG. 9 illustrates a gas analyzing method performed using a plurality of sensors 10 which exhibit mutually different molecular adsorption behaviors. Note that gas analyzing system 100 that performs the other example of operation illustrated in FIG. 9 need not include selector 33.

As illustrated in FIG. 9, exposer 20 exposes the plurality of sensors 10 to the sample gas only in the second period of the measurement period in the exposing step (Step S11). Next, in the obtaining step, obtainer 32 obtains a plurality of signals outputted from the plurality of sensors 10 exposed in Step S11 (Step S12). Steps S11 and S12 are identical to Steps S1 and S2, except that more than one sensor 10 is used in Steps S11 and S12.

Next, in the extracting step, extractor 34 extracts a plurality of features from the plurality of signals obtained in the obtaining step (Step S13). For example, extractor 34 extracts, for each of the plurality of signals: a difference between a signal value at a predetermined point in time after change due to the exposure of sensor 10 to the sample gas and a signal value before this change; and an amount of change in signal value per unit time (that is, the slope of the signal waveform) in a predetermined period. Note that extractor 34 may use any method to extract the plurality of features. Extractor 34 may extract the signal values as the features at predetermined intervals in measurement period Tm, for each of the plurality of signals. Alternatively, if deriving an approximate expression where the signal value is a time function, extractor 34 may extract a coefficient of the approximate expression as the feature.

Next, in the analyzing step, using the pre-trained logical model for determining the concentration of a predetermined molecule contained in the sample gas, analyzer 35 performs analysis by determining the concentration of the predetermined molecule contained in the sample gas, based on the plurality of features extracted by extractor 34 in the extracting step, and outputs an analysis result (Step S14). To be more specific, analyzer 35 may determine the concentration of water molecule as the predetermined molecule contained in the sample gas. For example, analyzer 35 may output a result indicating which one of a plurality of concentration candidates corresponds to the concentration of the predetermined molecule contained in the sample gas.

As described above, the gas analyzing method is used by gas analyzing system 100 including the plurality of sensors 10 each of which exhibits a different molecular adsorption behavior, for example. The gas analyzing method includes the obtaining step, the extracting step, and the analyzing step. In the obtaining step, the plurality signals outputted from the plurality of sensors 10 are obtained (Step S12). In the extracting step, the plurality of features are extracted from the plurality of signals (Step S13). In the analyzing step, the analysis by determining the concentration of the predetermined molecule contained in the sample gas is performed using the pre-trained logical model, based on the plurality of features extracted in the extracting step, and an analysis result is outputted (Step S14).

The signal outputted from sensor 10 changes in response to molecular adsorption to sensor 10. From the signal value outputted from sensor 10, the concentration of molecule contained in the sample gas can be determined. However, as compared to the case where the sample gas contains only the predetermined molecule, the signal value outputted from sensor 10 fluctuates when the sample gas contains a different molecule other than the analysis target molecule. This results in a decrease in the analytical accuracy. In contrast, the aforementioned gas analyzing method determines the concentration of the predetermined molecule contained in the sample gas, based on the plurality of features extracted from the signals outputted from the plurality of sensors 10 which exhibit mutually different molecular adsorption behaviors. Thus, even if the sample gas contains a different molecule other than the analysis target molecule, the use of the plurality of features extracted as described allows the effect of this different molecule on the analysis result to be reduced. Hence, the analytical accuracy can be increased even in the presence of a different molecule other than the analysis target molecule.

### EXAMPLE

The following describes the present disclosure in detail by way of example. Note that the example described below is not intended to limit the present disclosure.

### [Analysis-Signal Obtainment]

Signals outputted from 16 sensors 10 disposed in casing 21 were first obtained.

### (Sensors)

The sixteen sensors 10 differ from each other in material (or more specifically, resin material) used for the respective sensing parts. The material used for the sensing part included a resin material and conducting particles dispersed in the resin material.

### (Gas)

Nitrogen having a humidity of 0% was used as the reference gas. Five different kinds of sample gases A to E were used as the sample gases. Each of these sample gases was nitrogen that contained a different one of the following five different kinds of identification target molecules, which are odor-determination reference odors, at odor intensity 2. To be more specific, sample gases A to E contained the respective identification target molecules as below.
Sample gas A: β-phenylethyl alcohol (molecular weight of 122.16)
Sample gas B: Methylcyclopentenolone (molecular weight of 112.13)
Sample gas C: Isovaleric acid (molecular weight of 102.13)
Sample gas D: γ-undecalactone (molecular weight of 184.27)
Sample gas E: skatole (molecular weight of 131.17)

Sensors 10 were exposed to sample gases A to E. Here, the humidity was adjusted to 0%, 20%, 40%, 60%, and 80% for each of these sample gases.

### (Signal Obtainment)

Signal obtainment operation was performed as follows. The 16 sensors 10 were exposed to the reference gas for first period T1 of 30 seconds. After this, the sixteen sensors 10 were exposed to the sample gas for second period T2 of 30 seconds. After this, the sixteen sensors 10 were exposed to the reference gas for third period T3 of 30 seconds. In one obtainment operation performed as above, the sixteen sensors 10 were exposed to the sample gas and the reference gas in measurement period Tm and the signals outputted from the 16 sensors 10 (that is, one set of signals) were obtained. This signal obtainment operation was performed 100 times for each of sample gases A to E for each of the humidity conditions (that is, 0%, 20%, 40%, 60%, and 80%). More specifically, 100 sets of signals were obtained for each of the sample gases for each humidity condition. The signal obtainment operation was performed at a temperature of 23° C.

### [Division of Second Period and Third Period]

Second period T2 and third period T3 were divided into respective three sections, which were sections s21 to s23 and sections s31 to s33, equal in time length. More specifically, second period T2 was divided into section s21, section s22, and section s23 in order from the beginning, and third period T3 was divided into section s31, section s32, and section s33 in order from the beginning, as illustrated in FIG. 5.

### [Feature Extraction]

For each of the obtained signals in sections s21 to s23 and sections s31 to s33 divided as described above, the extracted features included: a difference between a signal value after change due to the exposure of sensor 10 to the sample gas and a signal value before this change; and an amount of change in signal value per unit time.

### Analysis Examples 1-1 to 1-4

In analysis examples 1-1 to 1-4, the analyses were performed using the features extracted as above to identify the kinds of molecules contained in the sample gases. Here, the features of the signals in the following sections (hereinafter, referred to as the analysis sections) were used.
Analysis example 1-1: all sections s21 to s23 and s31 to s33
Analysis example 1-2: section s21 and section s31
Analysis example 1-3: section s22 and section s32
Analysis example 1-4: section s23 and section s33

The logical model used in the analyses was a random-forest logical model that receives the feature extracted as above and outputs a result indicating one of the five identification target molecules.

For machine learning to build a pre-trained logical model used in analysis examples 1-1 to 1-4, the features of the signals in the analysis sections as a result of the exposure to sample gases A to E each having the humidity adjusted to 20% were used as learning data. The features of all the signals obtained in the analysis sections were inputted into the aforementioned pre-trained logical model to perform the analyses to identify the kinds of molecules contained in the sample gases. To be more specific, the analyses were performed to identify the kinds of molecules contained in the sample gases in humidity conditions different from the humidity condition used for the machine learning. The results of the analyses are shown in Table 1. Each accuracy rate in Table 1 is a rate of correctness of the identification target molecule outputted as the result of inputting, into the pre-trained logical model, the feature extracted using the corresponding one of sample gases A to E in the humidity condition indicated in the column “Test” in Table 1. The accuracy rate indicated in the row “Average” in Table 1 is an average of the accuracy rates of the analyses performed in the humidity conditions of 0%, 20%, 40%, 60%, and 80%. The same representation is used for accuracy rates in Table 2 below.

TABLE 1

| | | Accuracy rate [%] | | | |
|---|---|---|---|---|---|
| | | Analysis | Analysis | Analysis | Analysis |
| | | example | example | example | example |
| | | 1-1 | 1-2 | 1-3 | 1-4 |
| | | Sections | Sections | Sections | Sections |
| Humidity condition | | s21-s23 and | s21 and | s22 and | s23 and |
| Learning | Test | s31-s33 | s31 | s32 | s33 |
| 20% | 0% | 67.2 | 69.4 | 70.5 | 82.4 |
| | 20% | 100.0 | 100.0 | 100.0 | 100.0 |
| | 40% | 63.2 | 79.9 | 81.0 | 91.7 |
| | 60% | 16.2 | 41.6 | 51.0 | 87.5 |
| | 80% | 20.3 | 21.7 | 22.9 | 77.3 |
| | Average | 53.4 | 62.5 | 65.1 | 87.8 |

As shown in Table 1, analysis example 1-4 performed based on the features of the signals in the third sections, section s23 of second period T2 and section s33 of third period T3, had the highest accuracy rate. The accuracy rate of this analysis example is higher than the accuracy rate of analysis example 1-1 performed based on the features of the signals in all the sections, seconds s21 to s23 and s31 to s33. Thus, even for identifying the kind of molecule contained in the sample gas in the humidity condition different from the humidity condition used in the machine learning, it was confirmed that the analytical accuracy was increased by identifying the kind of molecule based on only the feature of the signal in one section from among the divided sections in second period T2 and third period T3 (for example, one section from among the second and subsequent sections from the beginning).

### Analysis Examples 2-1 to 2-4

In analysis examples 2-1 to 2-4, the analyses were performed using the features extracted as above to identify the kinds of molecules contained in the sample gases. Here, the features of the signals in the following analysis sections were used.
Analysis example 2-1: all sections s21 to s23 and s31 to s33
Analysis example 2-2: section s23 and section s33
Analysis example 2-3: section s23
Analysis example 2-4: section s33

The logical model used in the analyses was a random-forest logical model that receives the feature extracted as above and outputs a result indicating one of the five identification target molecules.

For machine learning to build a pre-trained logical model used in analysis examples 2-1 to 2-4, the features of the signals in the analysis sections as a result of the exposure to sample gases A to E each having the humidity adjusted to 40% were used as learning data. The features of all the signals obtained in the analysis sections were inputted into the aforementioned pre-trained logical model to perform the analyses to identify the kinds of molecules contained in the sample gases. To be more specific, the analyses were performed to identify the kinds of molecules contained in the sample gases in humidity conditions different from the humidity condition used for the machine learning. The results of the analyses are shown in Table 2.

TABLE 2

| | | Accuracy rate [%] | | | |
|---|---|---|---|---|---|
| | | Analysis | Analysis | | |
| | | example | example | Analysis | Analysis |
| | | 2-1 | 2-2 | example | example |
| | | Sections | Sections | 2-3 | 2-4 |
| Humidity condition | | s21-s23 and | s23 and | Section | Section |
| Learning | Test | s31-s33 | s33 | s23 | s33 |
| 40% | 0% | 43.5 | 62.7 | 64.6 | 66.1 |
| | 20% | 68.0 | 87.2 | 79.1 | 94.3 |
| | 40% | 100.0 | 100.0 | 100.0 | 100.0 |
| | 60% | 80.5 | 91.1 | 88.3 | 91.7 |
| | 80% | 43.2 | 74.2 | 59.8 | 71.5 |
| | Average | 67.0 | 83.0 | 78.4 | 84.7 |

As shown in Table 2, the accuracy rates of analysis examples 2-2 to 2-4 performed based on the features of the signals in the third section of second period T2 and/or the third section of third period T3 were higher than the accuracy rate of analysis example 2-1 performed based on the features of the signals in all the sections, seconds s21 to s23 and s31 to s33. Among the accuracy rates of analysis examples 2-2 to 2-4 performed based on the features of the signals in the third section of second period T2 and/or the third section of third period T3, the accuracy rate of analysis example 2-4 performed based on the feature of the signal in the last section, section s33, of third period T3 was the highest. Thus, for identifying the kind of molecule contained in the sample gas in the humidity condition different from the humidity condition used in the machine learning, it was confirmed that the analytical accuracy was further increased by identifying the kind of molecule based on only the feature of the signal in one section from among the divided sections in third period T3.

### Analysis Examples 3-1 to 3-4

In analysis examples 3-1 to 3-4, the analyses were performed using the features extracted as above to determine the concentrations of water molecule, that is, the humidities, in the sample gases. Here, the features of the signals in the following analysis sections were used.
Analysis example 3-1: all sections s21 to s23 and s31 to s33
Analysis example 3-2: section s21 and section s31
Analysis example 3-3: section s22 and section s32
Analysis example 3-4: section s23 and section s33

The logical model used in the analyses was a random-forest logical model that receives the feature extracted as above and outputs a result indicating one of 0%, 20%, 40%, 60%, and 80% as the humidity of the sample gas.

For machine learning to build a pre-trained logical model used in analysis examples 3-1 to 3-4, the features of the signals in the analysis sections as a result of the exposure to sample gas C having the humidity adjusted to 0%, 20%, 40%, 60%, and 80% were used as learning data. The features of all the signals obtained in the analysis sections were inputted into the aforementioned pre-trained logical model to perform the analyses to determine the humidities of the sample gases. To be more specific, the analyses were performed to identify the humidities of the sample gases containing molecules of odor-determination reference odors different from the molecule of odor-determination reference odor used for the machine learning. The results of the analyses are shown in Table 3. Each accuracy rate in Table 3 is a rate of correctness of the humidity outputted as the result of inputting, into the pre-trained logical model, the feature extracted when the corresponding one of the sample gases indicated in the column “Test” in Table 3 was adjusted to the corresponding humidity condition. The accuracy rate indicated in the row “Average” in Table 3 is an average of the accuracy rates of the analyses performed on sample gases A to E.

TABLE 3

| | | Accuracy rate [%] | | | |
|---|---|---|---|---|---|
| | | Analysis | Analysis | Analysis | Analysis |
| | | example | example | example | example |
| | | 3-1 | 3-2 | 3-3 | 3-4 |
| | | Sections | Sections | Sections | Sections |
| Sample gas | | s21-s23 and | s21 and | s22 and | s23 and |
| Learning | Test | s31-s33 | s31 | s32 | s33 |
| C | A | 76.0 | 93.8 | 84.4 | 47.1 |
| | B | 75.2 | 100.0 | 82.0 | 46.6 |
| | C | 100.0 | 100.0 | 100.0 | 100.0 |
| | D | 83.7 | 93.1 | 73.0 | 29.0 |
| | E | 79.9 | 100.0 | 95.1 | 51.0 |
| | Average | 83.0 | 97.4 | 86.9 | 54.7 |

As shown in Table 3, analysis example 3-2 performed based on the features of the signals in the first sections, section s21 of second period T2 and section s31 of third period T3, had the highest accuracy rate. The accuracy rate of this analysis example is higher than the accuracy rate of analysis example 3-1 performed based on the features of the signals in all the sections, seconds s21 to s23 and s31 to s33. Thus, even for determining the humidity of the sample gas containing the molecule of odor-determination reference odor different from the molecule of odor-determination reference odor used for the machine learning, it was confirmed that the analytical accuracy was increased by determining the humidity based on only the feature of the signal in one section from among the divided sections in each of second period T2 and third period T3 (for example, one section from among the second and preceding sections from the end).

### OTHER EMBODIMENTS

Although the gas analyzing system and the gas analyzing method according to aspects of the present disclosure have been described based on an embodiment, the present disclosure is not limited to this embodiment. Those skilled in the art will readily appreciate that embodiments arrived at by making various modifications to the above embodiment or embodiments arrived at by selectively combining elements disclosed in the above embodiment without materially departing from the scope of the present disclosure may be included within one or more aspects of the present disclosure.

Although exposer 20 exposes sensor 10 to the reference gas in first period T1 and third period T3, this is not intended to be limiting. Exposer 20 simply has to keep sensor 10 from being exposed to the sample gas in first period T1 and third period T3. For example, instead of exposing sensor 10 to the reference gas, exposer 20 may have the sample gas sucked out in order for sensor 10 to be exposed to a vacuum atmosphere.

Although selector 33 selects one extraction section from each of second period T2 and third period T3 in the selecting step according to the present embodiment, this is not intended to be limiting. Selector 33 may select a plurality of extraction sections from at least either one of second period T2 and third period T3.

Although gas analyzing system 100a includes detecting device 200 and analyzing device 300 for example, this is not intended to be limiting. Gas analyzing system 100a may include only analyzing device 300. In this case, Step S1 of FIG. 4 is omitted, for example. Then, obtainer 32a obtains, via the network, the signal already obtained by the sensor, for example.

It should be noted in the above-described embodiment that a part of all of the constituent elements included in the gas analyzing system according to the present disclosure may be implemented to a dedicated hardware, or implemented by executing a software program suitable for each of the constituent elements.

It should also be noted that the structural elements included in the gas analyzing system according to the present disclosure may be implemented to one or more electronic circuits. Each of the one or more electronic circuits may be a general-purpose circuit or a dedicated circuit.

The one or more electronic circuits may include, for example, a semiconductor device, an Integrated Circuit (IC) or a Large Scale Integration (LSI). The IC or LSI may be integrated to a single chip or integrated to a plurality of chips. Note that here, the terminology “LSI” or “IC” is used, but depending on the degree of integration, the circuit may also be referred to as a system LSI, a very large scale integration (VLSI), or an ultra large scale integration (ULSI). A field programmable gate array (FPGA) that is programed after manufacturing the LSI, or a reconfigurable logic device capable of reconfiguring the connections and settings of the circuit cells in the LSI may be used for the same purpose.

It should be noted that general or specific aspects of the present disclosure may be implemented to a system, a device, a method, an integrated circuit, or a computer program. The general or specific aspects of the present disclosure may be implemented to a non-transitory computer-readable recording medium such as an optical disk, a Hard Disk Drive (HDD), or a semiconductor memory, on which the computer program is recorded. The general or specific aspects of the present disclosure may be implemented to any given combination of a system, a device, a method, an integrated circuit, a computer program, or a recording medium.

For example, the present disclosure may be implemented to the gas analyzing method executed by a computer such as the gas analyzing system, or may be implemented to a program that causes a computer to execute such a gas analyzing method. Furthermore, the present disclosure may be implemented to a non-transitory computer-readable recording medium on which the above program is recorded,

The following describes the features of the gas analyzing system and the gas analyzing method described based on the above embodiment.
(1) A gas analyzing method using a sensor that outputs a signal responsive to molecular adsorption, the gas analyzing method comprising:
obtaining a signal outputted from the sensor that is exposed to a sample gas in a measurement period including a first period, a second period following the first period, and a third period following the second period, under a condition where a molecule contained in the sample gas is more readily adsorbed to the sensor in the second period than in the first period and the third period;
dividing at least one of the second period or the third period into a plurality of divided sections, and selecting, as at least one extraction section, a part of the plurality of divided sections;
extracting at least one feature of the signal outputted in the at least one extraction section; and
performing, using a pre-trained logical model for one of qualitative determination or quantitative determination of the molecule contained in the sample gas, one of qualitative analysis or quantitative analysis on the molecule contained in the sample gas, based on only the at least one feature extracted in the extracting among features of the signal in the measurement period, and outputting an analysis result of the performing of the one of qualitative analysis or quantitative analysis.
(2) The gas analyzing method according to (1),
wherein in the selecting, the at least one extraction section is selected from among a divided section counted second from a beginning of each of the second period and the third period and one or more divided sections subsequent to the divided section in each of the second period and the third period among the plurality of divided sections.
(3) The gas analyzing method according to (2),
wherein in the selecting, the at least one extraction section is selected from a last divided section in each of the second period and the third period among the plurality of divided sections.
(4) The gas analyzing method according to (2) or (3),
wherein in the selecting, the at least one extraction section is selected from divided sections in the third period among the plurality of divided sections.
(5) The gas analyzing method according to any one of (2) to (4),
wherein in the performing of the one of qualitative analysis or quantitative analysis, analysis to identify a kind of the molecule contained in the sample gas is performed.
(6) The gas analyzing method according to (5),
wherein in the performing of the one of qualitative analysis or quantitative analysis, analysis to identify a kind of an organic compound as the molecule contained in the sample gas is performed.
(7) The gas analyzing method according to (1),
wherein in the selecting, the at least one extraction section is selected from a divided section counted second from an end of each of the second period and the third period and one or more divided sections preceding the divided section in each of the second period and the third period among the plurality of divided sections.
(8) The gas analyzing method according to (7),
wherein in the selecting, the at least one extraction section is selected from a divided section counted first in each of the second period and the third period among the plurality of divided sections.
(9) The gas analyzing method according to one of (7) or (8),
wherein in the performing of the one of qualitative analysis or quantitative analysis, analysis to determine a concentration of a predetermined molecule contained in the sample gas is performed.
(10) The gas analyzing method according to (9),
wherein in the performing of the one of qualitative analysis or quantitative analysis, analysis to determine a concentration of a water molecule as the predetermined molecule contained in the sample gas is performed.
(11) The gas analyzing method according to any one of (1) to (10),
wherein the at least one feature includes at least one of: a difference between a value of the signal after change due to exposure of the sensor to the sample gas and a value of the signal before the change; or an amount of change in the value of the signal per unit time.
(12) The gas analyzing method according to any one of (1) to (11),
wherein in the obtaining, the signal outputted from the sensor exposed to the sample gas only in the second period of the measurement period is obtained.
(13) A gas analyzing method using a plurality of sensors each of which outputs a signal responsive to molecular adsorption, the plurality of sensors exhibiting mutually different molecular adsorption behaviors, the gas analyzing method comprising:
obtaining a plurality of signals outputted from each of the plurality of sensors that are exposed to a sample gas in a measurement period including a first period, a second period following the first period, and a third period following the second period, under a condition where a molecule contained in the sample gas is more readily adsorbed to the plurality of sensors in the second period than in the first period and the third period;
extracting a plurality of features from each of the plurality of signals; and
performing analysis to determine, using a pre-trained logical model for determining a concentration of a predetermined molecule contained in the sample gas, the concentration of the predetermined molecule contained in the sample gas, based on the plurality of features extracted in the extracting, and outputting an analysis result of the performing of analysis.
(14) The gas analyzing method according to (13),
wherein in the performing of analysis, a concentration of a water molecule as the predetermined molecule contained in the sample gas is determined.
(15) The gas analyzing method according to any one of (1) to (14),
wherein in the obtaining, the signal outputted from the sensor is obtained via a network.
(16) The gas analyzing method according to any one of (1) to (15),
wherein the sensor includes a sensitive membrane.
(17) The gas analyzing method according to (16),
wherein the sensitive membrane includes a resin material and conductive particles dispersed in the resin material.
(18) A gas analyzing system comprising:
a sensor that outputs a signal responsive to molecular adsorption;
an exposer that exposes the sensor to a sample gas in a measurement period including a first period, a second period following the first period, and a third period following the second period, under a condition where a molecule contained in the sample gas is more readily adsorbed to the sensor in the second period than in the first period and the third period;
an obtainer that obtains the signal outputted from the sensor in the measurement period;
a selector that divides at least one of the second period or the third period into a plurality of divided sections, and selects, as at least one extraction section, a part of the plurality of divided sections;
an extractor that extracts at least one feature of the signal outputted in the at least one extraction section;
a memory that stores a pre-trained logical model for one of qualitative determination or quantitative determination of the molecule contained in the sample gas; and
an analyzer that performs, using the pre-trained logical model, one of qualitative analysis or quantitative analysis on the molecules contained in the sample gas, based on only the at least one feature extracted by the extractor among features of the signal in the measurement period, and outputs an analysis result of the one of qualitative analysis or quantitative analysis.
(19) A gas analyzing system comprising:
a plurality of sensors each of which outputs a signal responsive to molecular adsorption, the plurality of sensors exhibiting mutually different molecular adsorption behaviors;
an exposer that exposes the plurality of sensors to a sample gas in a measurement period including a first period, a second period following the first period, and a third period following the second period, under a condition where a molecule contained in the sample gas is more readily adsorbed to the plurality of sensors in the second period than in the first period and the third period;
an obtainer that obtains a plurality of signals outputted from each of the plurality of sensors in the measurement period;
an extractor that extracts a plurality of features from each of the plurality of signals;
a memory that stores a pre-trained logical model for determining a concentration of a predetermined molecule contained in the sample gas; and
an analyzer that performs analysis to determine, using the pre-trained logical model, the concentration of the predetermined molecule contained in the sample gas, based on the plurality of features extracted by the extractor, and outputs an analysis result of the analysis.

### INDUSTRIAL APPLICABILITY

The gas analyzing system and the gas analyzing method according to the present disclosure are useful in qualitative analysis and quantitative analysis of a molecule contained in a gas.

1. A gas analyzing method using a sensor that outputs a signal responsive to molecular adsorption, the gas analyzing method comprising:
obtaining a signal outputted from the sensor that is exposed to a sample gas in a measurement period including a first period, a second period following the first period, and a third period following the second period, under a condition where a molecule contained in the sample gas is more readily adsorbed to the sensor in the second period than in the first period and the third period;
dividing at least one of the second period or the third period into a plurality of divided sections, and selecting, as at least one extraction section, a part of the plurality of divided sections;
extracting at least one feature of the signal outputted in the at least one extraction section; and
performing, using a pre-trained logical model for one of qualitative determination or quantitative determination of the molecule contained in the sample gas, one of qualitative analysis or quantitative analysis on the molecule contained in the sample gas, based on only the at least one feature extracted in the extracting among features of the signal in the measurement period, and outputting an analysis result of the performing of the one of qualitative analysis or quantitative analysis.

2. The gas analyzing method according to claim 1,
wherein in the selecting, the at least one extraction section is selected from among a divided section counted second from a beginning of each of the second period and the third period and one or more divided sections subsequent to the divided section in each of the second period and the third period among the plurality of divided sections.

3. The gas analyzing method according to claim 2,
wherein in the selecting, the at least one extraction section is selected from a last divided section in each of the second period and the third period among the plurality of divided sections.

4. The gas analyzing method according to claim 2,
wherein in the selecting, the at least one extraction section is selected from divided sections in the third period among the plurality of divided sections.

5. The gas analyzing method according to claim 2,
wherein in the performing of the one of qualitative analysis or quantitative analysis, analysis to identify a kind of the molecule contained in the sample gas is performed.

6. The gas analyzing method according to claim 5,
wherein in the performing of the one of qualitative analysis or quantitative analysis, analysis to identify a kind of an organic compound as the molecule contained in the sample gas is performed.

7. The gas analyzing method according to claim 1,
wherein in the selecting, the at least one extraction section is selected from a divided section counted second from an end of each of the second period and the third period and one or more divided sections preceding the divided section in each of the second period and the third period among the plurality of divided sections.

8. The gas analyzing method according to claim 7,
wherein in the selecting, the at least one extraction section is selected from a divided section counted first in each of the second period and the third period among the plurality of divided sections.

9. The gas analyzing method according to claim 7,
wherein in the performing of the one of qualitative analysis or quantitative analysis, analysis to determine a concentration of a predetermined molecule contained in the sample gas is performed.

10. The gas analyzing method according to claim 9,
wherein in the performing of the one of qualitative analysis or quantitative analysis, analysis to determine a concentration of a water molecule as the predetermined molecule contained in the sample gas is performed.

11. The gas analyzing method according to claim 1,
wherein the at least one feature includes at least one of: a difference between a value of the signal after change due to exposure of the sensor to the sample gas and a value of the signal before the change; or an amount of change in the value of the signal per unit time.

12. The gas analyzing method according to claim 1,
wherein in the obtaining, the signal outputted from the sensor exposed to the sample gas only in the second period of the measurement period is obtained.

13. (canceled)

14. (canceled)

15. The gas analyzing method according to claim 1,
wherein in the obtaining, the signal outputted from the sensor is obtained via a network.

16. The gas analyzing method according to claim 1,
wherein the sensor includes a sensitive membrane.

17. The gas analyzing method according to claim 16,
wherein the sensitive membrane includes a resin material and conductive particles dispersed in the resin material.

18. A gas analyzing system comprising:
a sensor that outputs a signal responsive to molecular adsorption;
an exposer that exposes the sensor to a sample gas in a measurement period including a first period, a second period following the first period, and a third period following the second period, under a condition where a molecule contained in the sample gas is more readily adsorbed to the sensor in the second period than in the first period and the third period;
an obtainer that obtains the signal outputted from the sensor in the measurement period;
a selector that divides at least one of the second period or the third period into a plurality of divided sections, and selects, as at least one extraction section, a part of the plurality of divided sections;
an extractor that extracts at least one feature of the signal outputted in the at least one extraction section;
a memory that stores a pre-trained logical model for one of qualitative determination or quantitative determination of the molecule contained in the sample gas; and
an analyzer that performs, using the pre-trained logical model, one of qualitative analysis or quantitative analysis on the molecules contained in the sample gas, based on only the at least one feature extracted by the extractor among features of the signal in the measurement period, and outputs an analysis result of the one of qualitative analysis or quantitative analysis.

19. (canceled)
